(19) United States
(12) Patent Application Publication
(10) Pub. No.: US 2024/0417675 A1
(43) Pub. Date: Dec. 19, 2024

(54) LACTOCOCCUS STRAINS

(71) Applicant: INTERNATIONAL N&H DENMARK APS, Kongens Lyngby (DK)

(72) Inventors: Elise Manoury, Chatellerault (FR); Pascal Fourcassie, Neuilly-sur-Seine (FR)

(21) Appl. No.: 18/580,960

(22) PCT Filed: Jul. 12, 2022
(86) PCT No.: PCT/EP2022/069446

§ 371 Date: Jan. 19, 2024

(30) Foreign Application Priority Data
Jul. 23, 2021 (EP) 21187394.8

(51) Int. Cl. C12N 1/20; A23C 11/10; A23L 11/50; C12R 1/46

(52) U.S. Cl. CPC C12N 1/205; A23C 11/10; A23C 11/106; A23L 11/50; A23V 2400/123; A23V 2400/231; A23V 2400/249; C12R 2001/46

(57) ABSTRACT

The present invention relates to bacterial strains of Lactococcus lactis or Lactococcus cremoris, usable as starter cultures, able to provide both satisfactory rheological and organoleptic proper-ties, and satisfactory shelf life to a plant-based media into which they are incorporated. The in-vention also provides a composition comprising one of these strains of Lactococcus lactis or Lactococcus cremoris, and feed or food products, in particular a plant-based feed or food product obtained with these strains.

### FIELD OF THE INVENTION

The present invention relates to bacterial strains of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, usable as starter cultures, able to provide both satisfactory rheological and organoleptic properties, and satisfactory shelf life to a plant-based media into which they are incorporated. The invention also provides a composition comprising one of these strains of of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, and feed or food products, in particular a plant-based feed or food product obtained with these strains.

### BACKGROUND OF THE INVENTION

The food industry uses bacteria in order to improve the taste and the texture of food or feed products. In the case of the dairy industry, lactic acid bacteria (LAB) are commonly used in order to, for example, bring about the acidification of milk (by fermentation) and to texturize the product into which they are incorporated. Among the lactic acid bacteria commonly used in the food industry, examples include the genera Streptococcus, Lactococcus, Lactobacillus, Leuconostoc, Pediococcus and Bifidobacterium.

The lactic acid bacteria are used in particular in the formulation of the starter cultures used for the production of fermented milks, for example yoghurts and for the manufacture of yoghurt and cheeses, such as Emmental, Gouda, Cheddar and Italian cheeses.

There is a need in the art for be able to prepare these dairy products from plant-based alternatives, a so-called plant-based dairy alternative product and accordingly there is a need for new and improved bacterial strains, in particular strains of species Lactococcus lactis or Lactococcus cremoris, which are useful for the fermentation of plant-based substrates with the same rheological or organoleptic properties, such as texture and flavor, of the food products as would be obtained with the known bacterial strains, and with a satisfactory shelf life to food or feed products.

### BRIEF DESCRIPTION OF THE DRAWINGS

FIG. 1. Acidification curves on soy milk at 40° C. Impact of addition of Lactococcus lactis DGCC1526 on acidification kinetic of Danisco® VEGE 31S during soy yoghurt preparation

FIG. 2. Acidification curves in oat 40° C. Impact of addition of Lactococcus lactis DGCC1526 on acidification kinetic of Danisco® VEGE 31S during Oat yoghurt preparation

FIG. 3. Acidification curves in soy milk inoculated with Danisco starter culture or Danisco starter culture and DSM33924 strain, temperature 37° C.

### SUMMARY OF THE INVENTION

It is an object of embodiments of the invention to provide bacterial strains suitable for the preparation of fermented plant-based dairy alternatives.

The present invention relates in a broad aspect to bacterial strains of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, which has the ability to acidify a plant-based substrate with the necessary speed and grow in plant-based preparations with a significant growth rate.

In another broad aspect, the present invention relates to the use of bacterial strains of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, for acidifying a plant-based substrate.

Accordingly, in a first aspect the present invention relates to a bacterial strain selected from the species of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, wherein the acidification kinetics of said bacterial strain in an aqueous preparation derived from a first plant containing 1-4% of protein from said plant and 2% glucose is characterized by:
an average time for reaching a pH of 6.00 less than about 510 min, measured as described in Assay I, such as less than 500 min, such as less than 490 min, such as less than 480 min, 470 min, 460 min, 450 min, 440 min, 430 min, 420 min, 410 min, 400 min, 390, or 380 min, and
an average time for reaching a pH of 5.5 less than about 660 min, measured as described in Assay I, such as less than 640 min, 620 min, 600 min, 580 min, 560 min, 540 min, 520 min, or 500 min.

In a second aspect the present invention relates to composition, such as a composition of a starter culture comprising or consisting of a one or more culture of a Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, and optionally further comprising at least one other microorganism, such as at least one other lactic acid bacterium and/or at least one propionic bacterium.

In a third aspect the present invention relates to the use of a culture of a bacterial strain according to the present invention or of a composition according to the present invention, such as a starter culture, for preparing a product, in particular a food or a feed product, such as a food product based on a plant, such as pea, soy, and oat, in particular a fermented product, in particular a fermented food or feed product based on a plant.

In a further aspect the present invention relates to a method for preparing a product, in particular a food or a feed product, such as based on a plant, such as pea, soy, and oat, in particular a fermented product, in particular a fermented food or feed product based on a plant, such as pea, soy, and oat, wherein said method comprises putting into contact a substrate, in particular a plant-milk substrate, with or in the presence of a culture of a bacterial strain according to the present invention or of a composition according to the present invention, optionally fermenting said substrate, and obtaining said product.

In a further aspect the present invention relates to a method for acidifying an aqueous preparation derived from a plant, which aqueous preparation contain 0-10%, such as 1-8%, or 2-7%, or 3-6% of protein from said plant and a sugar, such as a sugar selected from glucose, sucrose, and dextrose, such as the natural sugar of said plant, such as in an amount of 1-10%, such as 2-8%, such as 2-7%, such as 2-6%, such as 2-5%.

In a further aspect the present invention relates to a product, in particular a food or a feed product, in particular a fermented product, in particular a fermented food or a fermented feed product, in particular a fermented food or feed product based on a plant, such as pea, soy, and oat, obtainable by the method of the invention.

In some embodiments this plant mentioned above is selected from legumes, such as the seeds of legumes including beans, such as soybeans, peas, favabeans, chickpea, lentils, mung bean; nuts such as almond, coconut, cashew nut, Brazil nut, hazelnut, macadamia nut, pecan nut, pistachio and walnut; cereals and pseudo cereals such as wheat, corn/maize, oats, sorghum, rice, barley, millet, triticale, buckwheat, rye, teff; tuber such as cassava, potato, tapioca, arrowroot; oleaginous plants such as hemp, canola, rapeseed, and sunflowers.

In a further aspect the present invention relates to a non-dairy fermentation product comprising one or more proteins derived from a plant-based source and a bacterial strain according to the present invention.

### DETAILED DESCRIPTION OF THE INVENTION

The present inventors have identified strains of Lactococcus, in particular strains of Lactococcus lactis, having a surprisingly good acidification kinetics when acidifying a plant juice made from a water-based extracts of a plant also known as a “plant milk”. Surprisingly, the present inventors have found that Lactoccocus lactis subsp. lactis strains are well-adapted to the fermentation of e.g. pea plant-based raw material (3.7% pea protein) at 37° C., containing at least 2% glucose. Within a study of about 130 biodiverse strains-more than 11 genera and 20 species, in 3 plant-based yogurt-type models (based on pea, soy and oat), acidification profiles of Lactococcus lactis subsp. lactis strains are quite robust and less affected/variable than other LAB strains. The present inventors envision that strains of subspecies Lactococcus lactis subsp. hordniae, or of species Lactococcus cremoris, such as of subspecies Lactococcus cremoris subsp. tructuae or Lactococcus cremoris subsp. cremoris may be equally suitable according to the present invention.

This is particularly surprising in the pea model, where all 28 Lactococcus lactis subsp. lactis acidification profiles begin quite early: time to reach pH6 (ie delta pH of 1 compared to initial pH of the model) is less than 8 hours (range for 28 strains min: 6.35 h, max: 8.38 h) where as for the other species it is from 8.4 hours, and usually more than 9-10 hours and most of the time even much more.

Growth was also found to be remarkably high with Lactococcus lactis subsp. lactis strains whatever the plant-based model considered: when inoculated at 1.10E6 cfu/mL, a population of up to 1.10E8 cfu/mL is reached only after 6 hours of fermentation. Remarkably, none of the other tested species reached this level of population in the pea model.

For plant-based fermented foods production, the present invention may provide for a shorter production duration (less than 8 hours) at a mild temperature (27° C.-37° C., or up to 39° C., such as in the range of 30° C.-39° C.). This may decrease the energy spent for production, thus increasing the sustainability of the technology aligned with the requirement for sustainable process for industrial food production. This short production time may also allow for an increase in the plant-based fermented food manufacturer's productivity, offering more affordable food for consumers thereby increasing plant-based fermented food attractivity.

The consistency of acidification with the different plant-based material tested (Soy, Pea, Oat) may also be used to design robust starter cultures with excellent performances for the production of plant-based fermented food, such as food produced with legumes, cereals or mixed of both.

Besides the above, strains of Lactococcus, e.g. Lactococcus lactis subsp. lactis strains are expected to produce, during fermentation, metabolites of interest to i) control the texture of the fermented products, such as exopolysaccharides, ii) control the flavor of fermented products, such as by diacetyl/acetoin/3-methyl-butanal production, iii) increase the nutritional value by vitamin K2 production, and iv) limit the growth of Gram+ pathogen spoilage by nisin production.

Furthermore, strains of Lactococcus, such as species of Lactococcus lactis, such as Lactococcus lactis subsp. lactis used alone or in combination with other lactic acid bacteria strains in order to prepare fermented dairy alternative products is favorably impacting the odor or aroma of the fermented products. It can be noticed a decrease of sensory attributes related to off-notes as “bitter almond aroma”, “pea odour”, “soy aroma”, “cereal odour” or “cereal aroma”. This phenomenon is specifically perceived for fermented legumes preparations based on soy or based on pea.

Accordingly, the use of Lactococcus lactis subsp. lactis strains able to grow extensively in plant-based material will impact advantageously the sensory profile, the nutritional value and the safety of plant-based fermented food.

As used herein “an aqueous preparation derived from a plant” or “an aqueous preparation derived from a first plant” or “plant milk” refers to a plant juice made from a water-based plant extract, usually resembling the colour of milk. Plant milks are vegan beverages consumed as plant-based alternatives to dairy milk, and often provide a creamy mouthfeel. Among the plants used to manufacture plant milk, almond, soy, and coconut, such as coconut milk are the most pre-ferred plant milks worldwide. A “plant milk” may include “soy milk”, “oat milk”, or “pea milk”. The plant-milk may be extracts of plant material which have been treated, such as from leguminous plants (soya bean, chick pea, lentil and the like) or from oilseeds (colza, Canola oil, soya bean, sesame, cotton and the like), which extract contains proteins in solution or in colloidal suspension, which are coagulable by chemical action, by acid fermentation, and/or by heat. In some specific embodiments, the milk substrate is a mixture of a plant-milk and an animal milk(s). The terms “first plant” or “second plant”, or “third plant” is used herein simply to distinguish between different plants, so that a “first plant” or “second plant”, or “third plant” may be the same plant or different plants.

In some embodiments, an aqueous preparation derived from a plant, such as the first, second, or third plant is not derived from soy.

We hereby confirm that the depositor, DuPont Nutrition Biosciences ApS, Denmark has given his unreserved and irrevocable consent to the deposited materials being made available to the public. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC).

The composition of the invention, preferably when used as a starter culture, can be a pure culture or a mixed culture. Thus, a pure culture is defined as a culture wherein all or substantially all the culture consists of the same bacterial strain of Lactococcus lactis, or Lactococcus cremoris of the invention. In the alternative, a mixed culture is defined as a culture comprising several microorganisms, in particular comprising several bacterial strains, including the Lactococcus strain of the invention.

In a particular embodiment, the composition of the invention is or consists of a pure culture of a specific strain of Lactococcus lactis or Lactococcus cremoris as defined herein.

In another embodiment, the composition of the invention comprises, in addition to a culture of a specific strain of Lactococcus lactis or Lactococcus cremoris of the invention, at least one other microorganism. The term “microorganism” is defined herein as any organism that may be combined with the Lactococcus of the invention, in particular for use in the preparation of products according to the invention. The term “microorganism” encompasses yeasts, molds, and bacteria, such as lactic acid bacteria species, a bifidobacterium species, a brevibacterium species, and/or a propionibacterium species.

In a particular embodiment of mixed culture, the composition comprises, in addition to a culture of the Lactococcus of the invention, at least one culture of lactic acid bacteria and/or at least one other culture of propionic bacteria. Suitable lactic acid bacteria include strains of a Streptococcus species, a Lactobacillus species including Lactobacillus acidophilus, an Enterococcus species, a Pediococcus species, aLeuconostoc species, and an Oenococcus species or any combination thereof. Other lactic acid bacteria species include Leuconostoc sp., Streptococcus thermophilus, Lactobacillus delbrueckii subsp. bulgaricus, and Lactobacillus helveticus.

Thus, the invention is also directed to, as a particular embodiment, a composition as defined herein comprising or consisting of a culture of the Lactococcus of the invention, at least or exactly 1, 2, 3, 4, 5, 6, 7, 8, 9, or 10 strain(s) of the Lactococcus lactis or Lactococcus cremoris strains of the invention.

In a particular embodiment, the composition comprises or consists of a culture of the Lactococcus lactis or Lactococcus cremoris of the invention, and at least one, in particular at least or exactly 1, 2, 3, 4, 5, 6, 7, 8, 9, or 10 strain(s) different from the Lactococcus strain of the invention, such as one or several strain(s) of the species Streptococcus thermophilus, Lactobacillus delbrueckii subsp. bulgaricus, and/or one or several strain(s) of the species Streptococcus thermophilus and Lactobacillus helveticus and/or any combination thereof. In a particular embodiment, the composition comprises or consists of a culture of the strain of Lactococcus of the invention, such as one strain of species Lactococcus lactis or Lactococcus cremoris, and a strain of the species Streptococcus thermophilus and Lactobacillus delbrueckii subsp. bulgaricus. In another particular embodiment, the composition comprises or consists of a culture of the Lactococcus strain of the invention being two strains of the species Lactococcus lactis or Lactococcus cremoris, and a strain of the species Streptococcus thermophilus and Lactobacillus delbrueckii subsp. bulgaricus.

In a particular embodiment, the composition comprises or consists of a culture of the Lactococcus lactis or Lactococcus cremoris of the invention and a complex mixed starter culture.

In a particular embodiment of any composition defined herein, either as a pure or mixed culture, the composition further comprises at least one probiotic strain such as Bifidobacterium animalis subsp. lactis, Lactobacillus acidophilus, Lactobacillus paracasei, or Lactobacillus casei.

In a particular embodiment, the composition as defined herein, either as a pure or mixed culture as defined above, further comprises, in particular food acceptable, component(s), such as, but not limited to, cryoprotective agents (or cryoprotectants), boosters and/or common additives. By “component”, it is meant any molecule or solution which is not a microorganism as defined above. By way of example, cryoprotective agents include, cyclodextrin, maltitol, trehalose, sucrose, maltodextrine or combinations thereof. By way of example, boosters include nucleotides. By way of example, common additives include nutrients such as yeast extracts, sugars, and vitamins.

In a particular embodiment, the composition as defined herein, either as a pure or mixed culture as defined above, with or without additional component(s) is in a liquid, a frozen, or a dried-powder form, such as obtained after freeze-drying.

In a particular embodiment, the composition of the invention, either as a pure or mixed culture as defined above, with or without additional component(s), comprises the Lactococcus lactis or Lactococcus cremoris strain of the invention (and optionally at least one other microorganism) in a concentrated form (concentrate), including frozen or dried concentrates. Thus, the concentration of the Lactococcus lactis or Lactococcus cremoris strain of the invention within the composition is in the range of 10⁵ to 4×10¹² CFU (colony forming units) per gram of the composition, preferably 10⁷ to 10¹² CFU, and more preferably at least at least 10⁷, at least 10⁸, at least 10⁹, at least 10¹⁰ or at least 10¹¹ CFU/g of the composition.

The invention also provides the use of a culture of a Lactococcus lactis or Lactococcus cremoris strain of the invention or the use of a composition as defined herein, for preparing products, in particular food products or feed products, in particular fermented products, in particular fermented food products or fermented feed products, in particular a fermented food or feed product based on a plant. Thus, the invention also provides a method for preparing a product, preferably a food or feed product, wherein said method comprises a) putting a substrate into contact with or in the presence of a culture of the Lactococcus lactis or Lactococcus cremoris strain of the invention or a composition as defined herein [or mixing a substrate with a culture of the Lactococcus lactis or Lactococcus cremoris strain of the invention or a composition as defined herein, b) optionally fermenting said substrate, and c) obtaining said product. In a particular embodiment, the invention also provides a method for preparing a fermented product, preferably a fermented food or feed product, wherein said method comprises fermenting a substrate with or in the presence of a culture of the Lactococcus lactis or Lactococcus cremoris strain of the invention or a composition as defined herein, and obtaining said fermented product, in particular a fermented food or feed product based on a plant.

The invention is also directed to any product, which is prepared from a Lactococcus lactis or Lactococcus cremoris strain of the invention or a composition as defined herein, in particular by the methods disclosed herein, or which contains or comprises a Lactococcus lactis or Lactococcus cremoris strain of the invention or a composition as defined herein. In a particular embodiment, the invention provides a product, in particular a food or a feed product, in particular a fermented product, in particular a fermented food or a fermented feed product, in particular a fermented food or feed product based on a plant obtainable or obtained by methods as described herein. The invention also provides a product, in particular a food or a feed product, in particular a fermented product, in particular a fermented food or a fermented feed product based on a plant, comprising a culture of the Lactococcus lactis or Lactococcus cremoris strain of the invention or comprising a composition as defined herein.

Suitable products include, but are not limited to, a food, a foodstuff, a food ingredient, a food additive, a food supplement, a functional food, a feed, a nutritional supplement, or a probiotic supplement. According to the invention, by “food” it is meant a product that is intended for human consumption. According to the invention, by “feed” it is meant a product that is intended to feed an animal. As used herein the term “food ingredient” includes a formulation, which is or can be added to foods and includes formulations which can be used at low levels in a wide variety of products that require, for example, acidification. As used herein, the term “functional food” means a food which is capable of providing not only a nutritional effect and/or a taste satisfaction but is also capable of delivering a further beneficial effect to the consumer. Suitable products include, but are not limited to, fruits, vegetables, fodder crops and vegetables including derived products, grain and grain-derived products, dairy foods and dairy food-derived products, meat, poultry, and seafood. The Lactococcus lactis or Lactococcus cremoris strain of the invention or a composition as defined herein can be used in the preparation of food products such as one or more of confectionery products, dairy products, meat products, poultry products, fish products and bakery products. By way of example, the Lactococcus lactis or Lactococcus cremoris strain of the invention or a composition as defined herein can be used as ingredients to a soft drink, a fruit juice or a beverage comprising whey protein, health tea, cocoa drink, milk drink and lactic acid bacteria drink, yoghurt, drinking yoghurt, and wine.

In a particular embodiment, the substrate into which the Lactococcus lactis or Lactococcus cremoris strain of the invention or a composition as defined herein is added to—or mixed with—is a plant-milk substrate. Therefore, in a particular embodiment, the invention also provides the use of a culture of a Lactococcus lactis or Lactococcus cremoris strain of the invention or the use of a composition as defined herein, for preparing a non-dairy plant-based product, in particular non-dairy plant-based food product or non-dairy plant-based feed product, in particular fermented non-dairy plant-based product, in particular fermented non-dairy plant-based food product or fermented non-dairy plant-based feed product. Thus, the invention also provides a method for preparing a non-dairy plant-based product, in particular non-dairy plant-based food product or non-dairy plant-based feed product, in particular fermented non-dairy plant-based product, in particular fermented non-dairy plant-based food product or fermented non-dairy plant-based feed product, wherein said method comprises a) putting into contact plant-milk substrate with or in the presence of a culture of the Lactococcus lactis or Lactococcus cremoris strain of the invention or with a composition as defined herein, b) optionally fermenting said plant-milk substrate and c) obtaining said product. In a particular embodiment, the invention also provides a method for preparing a fermented non-dairy plant-based product, preferably fermented non-dairy plant-based food or feed product, wherein said method comprises fermenting plant-milk substrate with or in the presence of a culture of the Lactococcus lactis or Lactococcus cremoris strain of the invention or a composition as defined herein and obtaining said fermented non-dairy plant-based product. In a particular embodiment, the plant-milk substrate comprises solid items, such as fruits, chocolate products, or cereals. In a particular embodiment, the invention is also directed to the use of the Lactococcus lactis or Lactococcus cremoris strain of the invention or any composition as defined herein (pure or mixed culture) to reduce the post-acidification phenomenon of the non-dairy plant-based product obtained with or of the non-dairy plant-based product fermented with or in presence of said Lactococcus lactis or Lactococcus cremoris strain or said composition, as compared to non-dairy plant-based product(s) obtained without or fermented without or in the absence of the Lactococcus lactis or Lactococcus cremoris strain of the invention, and directed to the non-dairy plant-based products per se. In a particular embodiment, the invention is also directed to the use of the Lactococcus lactis or Lactococcus cremoris strain of the invention or any composition as defined herein (pure or mixed culture) to obtain a non-dairy plant-based product, in particular a plant-based yoghurt, whose pH is 4.4±0.1 and is stable when the product is stored during 14 days at a positive temperature less than 10° C. In a particular embodiment, the invention is also directed to the use of the Lactococcus lactis or Lactococcus cremoris strain of the invention or any composition as defined herein (pure or mixed culture) to obtain a non-dairy plant-based product, in particular a plant-based yoghurt, whose pH is 4.5±0.1 or is 4.4±0.05 when the non-dairy plant-based product is stored during 14 days at a positive temperature less than 10° C., and optionally whose pH is stable (i.e, within the same range) until 28 days.

Therefore, the invention provides a non-dairy plant-based product, in particular non-dairy plant-based food product or non-dairy plant-based feed product, in particular fermented non-dairy plant-based product, in particular fermented non-dairy plant-based food product or fermented non-dairy plant-based feed product, obtainable or obtained by methods as described herein with a plant-milk substrate. The invention also provides a non-dairy plant-based product, in particular a fermented non-dairy plant-based product comprising a culture of the Lactococcus lactis or Lactococcus cremoris strain of the invention or comprising a composition as defined herein. In a particular embodiment, the non-dairy plant-based product or fermented non-dairy plant-based product is or comprises a plant-based yoghurt, a plant-based cheese (such as an acid plant-based curd cheese, a hard plant-based cheese, a semi-hard plant-based cheese, a plant-based cottage cheese), plant-based quark, plant-based kefir, a plant-based koumiss, a plant-based yoghurt drink, a plant-based fromage frais, a plant-based cottage cheese, a plant-based cream dessert.

As detailed above the present invention relates to a bacterial strain selected from the species of Lactococcus lactis or Lactococcus cremoris, wherein the acidification kinetics of the bacterial strain in an aqueous preparation derived from a first plant containing 1-4% of protein from said plant and 2% glucose is characterized by:
an average time for reaching a pH of 6.00 less than about 510 min, measured as described in Assay I, such as less than 500 min, such as less than 490 min, such as less than 480 min, 470 min, 460 min, 450 min, 440 min, 430 min, 420 min, 410 min, 400 min, 390, or 380 min, and
an average time for reaching a pH of 5.5 less than about 660 min, measured as described in Assay I, such as less than 640 min, 620 min, 600 min, 580 min, 560 min, 540 min, 520 min, or 500 min.

In some specific embodiments the bacterial strain is the species of Lactococcus lactis, which species may also be referred to as Lactoccocus lactis lactis.

In some embodiments the growth of the bacterial strain in an aqueous preparation derived from a second plant containing 1-4% of protein from said plant and 2% glucose measured as described in Assay II is characterized by:
an average of at least about 1.5×108 cfu/ml after 6 hours of incubation in the aqueous preparation derived from a plant from an inoculation with 1×106 cfu/ml.

In some embodiments the bacterial strain bacterial strain originates from fermentation in an aqueous preparation derived from a third plant.

It is to be understood that when referring to a first, second and third plant this means that they may be the same plant, or they may be different plants. Accordingly, a specific strain of the invention may derive from or be used in the fermentation of one plant, whereas the selection based on acidification kinetics and/or growth is based on tests in an aqueous preparation derived from another (or the same) plant.

Thus, in some embodiments the two or three of said first, second and third plant are the same plant.

In some embodiments the two or three of said first, second and third plant are different plants. In some embodiments the first, second and/or third plant is independently selected from legumes, such as the seeds of legumes including beans, such as soybeans, peas, favabeans, chickpea, lentils, mung bean; nuts such as almond, coconut, cashew nut, Brazil nut, hazelnut, macadamia nut, pecan nut, pistachio and walnut; cereals and pseudo cereals such as wheat, corn/maize, oats, sorghum, rice, barley, millet, triticale, buckwheat, rye, teff; tuber such as cassava, potato, tapioca, arrowroot; oleaginous plants such as hemp, canola, rapeseed, and sunflowers.

In some embodiments the first, second and/or third plant is independently selected from pea, soy, and oat.

In some embodiments the bacterial strain of the invention is a protease-negative bacterial strain.

In some embodiments the bacterial strain of the invention is a strain of subspecies Lactococcus lactis subsp. lactis.

In some embodiments the bacterial strain of the invention is selected from the group consisting of:
(1) the DSM 33924 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(2) the DSM 33925 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(3) the DSM 33926 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(4) the DSM 33927 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(5) the DSM 33928 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(6) the DSM 33929 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH; and
(7) a mutant bacterial strain of the DSM 33924 strain, the DSM 33925 strain, the DSM 33926 strain, the DSM 33927 strain, the DSM 33928 strain, or the DSM 33929 strain.

One aspect of the invention relates a to composition comprising or consisting of a one or more culture of a bacterial strain of the invention, and optionally further comprising at least one other microorganism, such as at least one other lactic acid bacterium and/or at least one propionic bacterium.

In some embodiments the at least one other lactic acid bacterium is a different bacterial strain of the species Lactoccocus lactis, a different bacterial strain of the species Lactococcus cremoris, a different bacterial strain of the species Lactococcus hordinae, a bacterial strain of the species Streptococcus thermophilus, and/or a bacterial strain of the species Lactobacillus, such as a bacterial strain of the subspecies Lactobacillus delbrueckii subsp. bulgaricus, and/or a bacterial strain of the genus Bifidobacterium, and/or any combination thereof.

In some embodiments the composition of the invention comprises or consist of a one or more culture of a bacterial strain of the invention, a bacterial strain of the species Streptococcus thermophilus, and a bacterial strain of Lactobacillus delbrueckii subsp. bulgaricus.

In some embodiments the composition of the invention is further comprising, in particular food acceptable, component(s) such as cryoprotective agents, and/or common additives.

In some embodiments the composition of the invention is in a liquid, a frozen or a dried-powder form.

In some embodiments the composition of the invention comprises or consist of one or more culture of a bacterial strain of the invention and no other bacterial strain.

In some embodiments the composition of the invention is comprises or consist of one or more culture of a bacterial strain Lactoccocus lactis of the invention and no other bacterial strain.

Numbered embodiments of the invention:
1. A bacterial strain selected from the species of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, wherein the acidification kinetics of said bacterial strain in an aqueous preparation derived from a first plant containing 1-4% of protein from said plant and 2% glucose is characterized by:
   an average time for reaching a pH of 6.00 less than about 510 min, measured as described in Assay I, such as less than 500 min, such as less than 490 min, such as less than 480 min, 470 min, 460 min, 450 min, 440 min, 430 min, 420 min, 410 min, 400 min, 390, or 380 min, and
   an average time for reaching a pH of 5.5 less than about 660 min, measured as described in Assay I, such as less than 640 min, 620 min, 600 min, 580 min, 560 min, 540 min, 520 min, or 500 min.
2. The bacterial strain according to embodiment 1, wherein the growth of said bacterial strain in an aqueous preparation derived from a second plant containing 1-4% of protein from said plant and 2% glucose measured as described in Assay II is characterized by:
   an average of at least about 1.5×10⁸ cfu/ml after 6 hours of incubation in the aqueous preparation derived from a plant from an inoculation with 1×10⁶ cfu/ml.
3. The bacterial strain according to any one of embodiments 1 or 2, wherein said bacterial strain originates from fermentation in an aqueous preparation derived from a third plant.
4. The bacterial strain according to any one of embodiments 1-3, wherein two or three of said first, second and third plant are the same plant.
5. The bacterial strain according to any one of embodiments 1-3, wherein two or three of said first, second and third plant are different plants.
6. The bacterial strain according to any one of embodiments 1-5, wherein said first, second and/or third plant is independently selected from legumes, such as the seeds of legumes including beans, such as soybeans, peas, favabeans, chickpea, lentils, mung bean; nuts such as almond, coconut, cashew nut, Brazil nut, hazelnut, macadamia nut, pecan nut, pistachio and walnut; cereals and pseudo cereals such as wheat, corn/maize, oats, sorghum, rice, barley, millet, triticale, buckwheat, rye, teff; tuber such as cassava, potato, tapioca, arrowroot; oleaginous plants such as hemp, canola, rapeseed, and sunflowers.
7. The bacterial strain according to any one of embodiments 1-6, wherein said first, second and/or third plant is independently selected from pea, soy, and oat.
8. The bacterial strain according to any one of embodiments 1-7, which is a protease-negative bacterial strain.
9. The bacterial strain according to any one of embodiments 1-8, which is a strain of subspecies Lactococcus lactis subsp. lactis.
10. The bacterial strain according to any one of embodiments 1-9, which is selected from the group consisting of:
   (1) the DSM 33924 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
   (2) the DSM 33925 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
   (3) the DSM 33926 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
   (4) the DSM 33927 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
   (5) the DSM 33928 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
   (6) the DSM 33929 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH; and
   (7) a mutant bacterial strain of the DSM 33924 strain, the DSM 33925 strain, the DSM 33926strain, the DSM 33927 strain, the DSM 33928 strain, or the DSM 33929 strain.
11. The bacterial strain according to any one of embodiments 1-10, wherein the acidification kinetics is measured at a temperature below 39° C., such as below 37° C., such as below 35° C., such as below 33° C. or even below 30° C., such as in the range of 27° C.-37° C., or in the range of 27° C.-39° C., such as in the range of 30° C.-39° C.
12. A composition, such as a composition of a starter culture comprising or consisting of a one or more culture of a bacterial strain as defined in any one of embodiments 1-11, and optionally further comprising at least one other microorganism, such as at least one other lactic acid bacterium and/or at least one propionic bacterium.
13. The composition according to embodiment 12, wherein said at least one other lactic acid bacterium is a different bacterial strain of the species Lactoccocus lactis, a different bacterial strain of the species Lactococcus cremoris, a different bacterial strain of the species Lactococcus hordinae, a bacterial strain of the species Streptococcus thermophilus, and/or a bacterial strain of the species Lactobacillus, such as a bacterial strain of the subspecies Lactobacillus delbrueckii subsp. bulgaricus, and/or a bacterial strain of the genus Bifidobacterium, and/or any combination thereof.
14. The composition according to any one of embodiments 12 or 13, which is comprising or consisting of a one or more culture of a bacterial strain as defined in any one of embodiments 1-11, a bacterial strain of the species Streptococcus thermophilus, and a bacterial strain of Lactobacillus delbrueckii subsp. bulgaricus.
15. The composition according to any one of embodiments 12-14, further comprising, in particular food acceptable, component(s) such as cryoprotective agents, and/or common additives.
16. The composition according to any one of embodiments 12-15, which is in a liquid, a frozen or a dried-powder form.
17. The composition according to any one of embodiments 12, 14-16, which composition comprises or consist of one or more culture of a bacterial strain as defined in any one of embodiments 1-11 and no other bacterial strain.
18. The composition according to any one of embodiments 12-17, which composition comprises or consist of one or more culture of a bacterial strain Lactoccocus lactis as defined in any one of embodiments 1-11 and no other bacterial strain.
19. Use of a culture of a bacterial strain selected from the species of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris, such as a starter culture, for preparing a product, in particular a food or a feed product, such as a food product based on a plant, such as pea, soy, and oat, in particular a fermented product, in particular a fermented food or feed product based on a plant.
20. Use of a culture of a bacterial strain as defined in any one of embodiments 1-11 or of a composition as defined in any one of embodiments 12-18, such as a starter culture, for preparing a product, in particular a food or a feed product, such as a food product based on a plant, such as pea, soy, and oat, in particular a fermented product, in particular a fermented food or feed product based on a plant.
21. The use according to any one of embodiments 19 or 20, wherein said plant is selected from legumes, such as the seeds of legumes including beans, such as soybeans, peas, favabeans, chickpea, lentils, mung bean; nuts such as almond, coconut, cashew nut, Brazil nut, hazelnut, macadamia nut, pecan nut, pistachio and walnut; cereals and pseudo cereals such as wheat, corn/maize, oats, sorghum, rice, barley, millet, triticale, buckwheat, rye, teff; tuber such as cassava, potato, tapioca, arrowroot; oleaginous plants such as hemp, canola, rapeseed, and sunflowers.
22. A method for preparing a product, in particular a food or a feed product, such as based on a plant, such as pea, soy, and oat, in particular a fermented product, in particular a fermented food or feed product based on a plant, such as pea, soy, and oat, wherein said method comprises putting into contact a substrate, in particular a plant-milk substrate, with or in the presence of a culture of a bacterial strain selected from the species of Lactococcus lactis, such as of subspecies Lactococcus lactis subsp.lactis or Lactococcus lactis subsp. hordniae, or of species Lactococcus cremoris, such as of subspecies Lactococcus cremoris subsp. tructuae or Lactococcus cremoris subsp. cremoris, optionally fermenting said substrate, and obtaining said product.
23. A method for preparing a product, in particular a food or a feed product, such as based on a plant, such as pea, soy, and oat, in particular a fermented product, in particular a fermented food or feed product based on a plant, such as pea, soy, and oat, wherein said method comprises putting into contact a substrate, in particular a plant-milk substrate, with or in the presence of a culture of a bacterial strain as defined in any one of embodiments 1-11 or of a composition as defined in any one of embodiments 12-18, optionally fermenting said substrate, and obtaining said product.
24. A method for acidifying an aqueous preparation derived from a plant, such as pea, soy, and oat, which aqueous preparation contain 0-10%, such as 1-8%, or 2-7%, or 3-6% of protein from said plant and a sugar, such as a sugar selected from glucose, sucrose, and dextrose, such as the natural sugar of said plant, such as in an amount of 1-10%, such as 2-8%, such as 2-7%, such as 2-6%, such as 2-5%, the method comprising putting into contact the aqueous preparation, in particular a plant-milk substrate, with or in the presence of a culture of a bacterial strain selected from the species of Lactococcus lactis, such as of subspecies Lactococcus lactis subsp. lactis or Lactococcus lactis subsp. hordniae, or of species Lactococcus cremoris, such as of subspecies Lactococcus cremoris subsp. tructuae or Lactococcus cremoris subsp. cremoris.
25. A method for acidifying an aqueous preparation derived from a plant, such as pea, soy, and oat, which aqueous preparation contain 0-10%, such as 1-8%, or 2-7%, or 3-6% of protein from said plant and a sugar, such as a sugar selected from glucose, sucrose, and dextrose, such as the natural sugar of said plant, such as in an amount of 1-10%, such as 2-8%, such as 2-7%, such as 2-6%, such as 2-5%, the method comprising putting into contact the aqueous preparation, in particular a plant-milk substrate, with or in the presence of a culture of a bacterial strain as defined in any one of embodiments 1-11 or of a composition as defined in any one of embodiments 12-18.
26. The method according to any one of embodiments 22-25, wherein said plant is selected from legumes, such as the seeds of legumes including beans, such as soybeans, peas, favabeans, chickpea, lentils, mung bean; nuts such as almond, coconut, cashew nut, Brazil nut, hazelnut, macadamia nut, pecan nut, pistachio and walnut; cereals and pseudo cereals such as wheat, corn/maize, oats, sorghum, rice, barley, millet, triticale, buckwheat, rye, teff; tuber such as cassava, potato, tapioca, arrowroot; oleaginous plants such as hemp, canola, rapeseed, and sunflowers.
27. A product, in particular a food or a feed product, in particular a fermented product, in particular a fermented food or a fermented feed product, in particular a fermented food or feed product based on a plant, such as pea, soy, and oat, obtainable by the method of embodiment 22-26.
28. A product, in particular a food or a feed product, in particular a fermented product, in particular a fermented food or a fermented feed product, in particular a fermented food or feed product based on a plant, such as pea, soy, and oat, comprising a culture of a bacterial strain as defined in any one of embodiments 1-11 or the composition as defined in any one of embodiments 12-18.
29. A product according to any one of embodiments 27 or 28, which is a plant-based dairy alternative product, in particular a product based on a plant, such as pea, soy, and oat, such as a plant-based yoghurt, cheese, quark, a sour cream, kefir, a koumiss, plant-based butter, a plant-milk beverage, a yoghurt plant drink, a fermented plant milk, a matured cream, a fromage frais, a cottage cheese, or a cream dessert.
30. A non-dairy fermentation product comprising one or more proteins derived from a plant-based source and a bacterial strain as defined in any one of embodiments 1-11.
31. A composition according to any one of embodiments 12-18, or a product, such as a non-dairy fermentation product according to any one of embodiments 27-30, wherein the culture of a bacterial strain as defined in any one of embodiments 1-11 is present in an amount of at least about 1×10⁶ cfu/ml.

### Analytical Methods

### Assay I

The acidification kinetics of fermentation with a bacterial strain assessed with an aqueous preparation derived from a plant, such as a pea protein preparation.

A plant-based preparation, such as pea-, soy or oat-based preparation, is made using plant protein isolates such as TRUPRO® 2000 for Pea Protein (IFF, Nutrition & Biosciences). A plant protein isolate is reconstituted in water to reach a final concentration in the range of 1-4%, such as 3.7-3.9% protein. Glucose is added to the preparation to achieve a concentration of 2% glucose. The preparation is thoroughly mixed for 10 minutes with a Thermomix at a temperature of 80° C. prior to pasteurization at 90° C. for 90 minutes. The preparation is stored overnight at refrigerated temperature (i.e. 4° C.). Before inoculation, the plant-based preparation is warmed to 37° C. Inoculation rate is 1E6 cfu/ml. The pH of the plant preparation is monitored during fermentation at 37° C. using an online pH measurement device (Abscia from Absciss Instrumenta- tion Scientifique) and the time to reach a pH of 6.00 (T PH 6.00 in minutes) and 5.5 (TpH 5.50 in minutes) is determined.

### Assay II

A plant-based preparation, such as a pea-, soy or oat-based preparation, is made like for Assay I. Before inoculation, the preparations are warmed to 37° C. Inoculation rate is 1E6 cfu/ml. After 6 hours of incubation the concentration in CFU/g of plant-based preparation is measured by using standard plate count technique.

### Examples 1

### Example 1: Acidification of Pea Protein Preparations Using Lactic Acid Bacteria

The acidification kinetics of pea protein preparations fermented with different lactic acid bacteria were assessed.

A pea-based preparation was made using pea protein isolates TRUPRO® 2000 Pea Protein (IFF, Nutrition & Biosciences). TRUPRO 2000 powder was reconstituted at 4.5% (W/W) in water to reach e a final concentration of 3.7% protein. Glucose was added to the preparation to achieve a concentration of 2% glucose. The preparation was thoroughly mixed for 10 minutes with a Thermomix at a temperature of 80° C. prior to pasteurization at 90° C. for 90 minutes. The preparation was stored overnight at refrigerated temperature (ie. 4° C.).

Before inoculation, the pea preparation was warmed to 37° C. The inoculation rate was 1E6 cfu/ml for each bacterial stain tested. The pH of the pea preparation was monitored during fer- mentation at 37° C. using an online pH measurement device (Abscia from Absciss Instrumentation Scientifique) and the time to reach a pH of 6.00 (T PH 6,00 in minutes) and 5.5 (TpH 5.50 in minutes) was determined.

The lactic acid bacteria tested included biodiverse Lactococcus lactis (28 strains) Streptococcus thermophilus (6 strains), Lacticaseibacillus paracaseï (23 strains), Lactiplantbacillus plantarum (7 strains), Lactiplantbacillus pentosus (15 strains), Levilactobacillus brevis (14 strains), Limosilactobacillus fermentum (9 strains), and Pediococus pentosaceus (3 strains). Table 1 shows the time to reach pH 6.00 (T PH 6,00 in minutes) and pH 5,5 (TpH 5.50 in minutes) for each of the 105 strains tested.

TABLE 1
TpH 6.00 and T PH 5.5 for 104 different Lactic acid bacteria
strain measured during pea preparation fermentation.

| | | tpH | T Ph |
|---|---|---|---|
| | | 6.00 | 5.50 |
| ID number | species | (min) | (min) |
| DSM33928 | Lactococcus lactis | 381 | 456 |
| DGCC12658 | Lactococcus lactis | 392 | 485 |
| DGCC12654 | Lactococcus lactis | 388 | 489 |
| DGCC12689 | Lactococcus lactis | 414 | 499 |
| DGCC12673 | Lactococcus lactis | 409 | 500 |
| DGCC12667 | Lactococcus lactis | 419 | 501 |
| DGCC12678 | Lactococcus lactis | 411 | 506 |
| DGCC7283 | Lactococcus lactis | 421 | 507 |
| DGCC12665 | Lactococcus lactis | 411 | 509 |
| DGCC12657 | Lactococcus lactis | 404 | 510 |
| DSM33924 | Lactococcus lactis | 408 | 515 |
| DGCC12686 | Lactococcus lactis | 430 | 519 |
| DGCC12682 | Lactococcus lactis | 413 | 531 |
| DSM33925 | Lactococcus lactis | 431 | 537 |
| DGCC12660 | Lactococcus lactis | 421 | 537 |
| DSM33927 | Lactococcus lactis | 446 | 540 |
| DGCC12663 | Lactococcus lactis | 425 | 545 |
| DGCC12691 | Lactococcus lactis | 448 | 548 |
| DGCC12662 | Lactococcus lactis | 451 | 554 |
| DSM33926 | Lactococcus lactis | 436 | 554 |
| DGCC12688 | Lactococcus lactis | 482 | 579 |
| DGCC2631 | Lactococcus lactis | 441 | 582 |
| DGCC12676 | Lactococcus lactis | 472 | 601 |
| DGCC12671 | Lactococcus lactis | 462 | 607 |
| DGCC12675 | Lactococcus lactis | 501 | 617 |
| DGCC12651 | Lactococcus lactis | 503 | 619 |
| DGCC12668 | Lactococcus lactis | 466 | 620 |
| DGCC12685 | Lactococcus lactis | 475 | 625 |
| TCF151MRS-A11 | Lactiplantibacillus pentosus | 551 | 639 |
| DGCC7710 | Streptococcus thermophilus | 506 | 640 |
| DGCC7666 | Streptococcus thermophilus | 594 | 680 |
| SC-19-D3 | Lactiplantibacillus pentosus | 598 | 690 |
| 14655 | Lacticaseibacillus paracasei | 613 | 694 |
| 6512 | Levilactobacillus brevis | 643 | 714 |
| TCF077MRS-A2 | Lactiplantibacillus pentosus | 638 | 714 |
| SC-26-H3 | Lactiplantibacillus pentosus | 635 | 732 |
| SC-20-H1 | Lactiplantibacillus pentosus | 669 | 738 |
| DGCC13615 | Lacticaseibacillus paracasei | 642 | 740 |
| TCF156MRS-A1 | Lactiplantibacillus pentosus | 643 | 745 |
| 14693 | Lacticaseibacillus paracasei | 661 | 751 |
| 14694 | Lacticaseibacillus paracasei | 683 | 774 |
| TCF149MRS-A1 | Lactiplantibacillus pentosus | 694 | 779 |
| 7393 | Lacticaseibacillus paracasei | 711 | 782 |
| DGCC7693 | Streptococcus thermophilus | 701 | 786 |
| TCF022LA-A12 | Lactiplantibacillus pentosus | 719 | 809 |
| 7669 | Lacticaseibacillus paracasei | 723 | 824 |
| DGCC7919 | Streptococcus thermophilus | 738 | 840 |
| DGCC8006 | Streptococcus thermophilus | 750 | 842 |
| 12689 | Lacticaseibacillus paracasei | 724 | 842 |
| 7681 | Lacticaseibacillus paracasei | 742 | 847 |
| 14634 | Lacticaseibacillus paracasei | 745 | 849 |
| DGCC4715 | Lactiplantibacillus plantarum | 749 | 853 |
| 14705 | Lacticaseibacillus paracasei | 754 | 859 |
| 7393 | Lacticaseibacillus paracasei | 771 | 889 |
| TCF049MRS-E9 | Lactiplantibacillus pentosus | 773 | 890 |
| 14687 | Lacticaseibacillus paracasei | 802 | 893 |
| 15321 | Lacticaseibacillus paracasei | 807 | 940 |
| 13394 | Lacticaseibacillus paracasei | 795 | 960 |
| DGCC6297 | Streptococcus thermophilus | 617 | 968 |
| 12726 | Lactiplantibacillus plantarum | 803 | 992 |
| DGCC13620 | Lacticaseibacillus paracasei | 868 | 1012 |
| 7387 | Lactiplantibacillus plantarum | 792 | 1034 |
| 13889 | Lacticaseibacillus paracasei | 924 | 1057 |
| 7694 | Lacticaseibacillus paracasei | 929 | 1062 |
| 14679 | Lacticaseibacillus paracasei | 992 | 1070 |
| 15338 | Lactiplantibacillus plantarum | 881 | 1105 |
| 11890 | Lacticaseibacillus paracasei | 970 | 1115 |
| DGCC13613 | Lactiplantibacillus plantarum | 960 | 1186 |
| DGCC11936 | Lactiplantibacillus pentosus | 939 | 1207 |
| 7682 | Lacticaseibacillus paracasei | 1116 | 1247 |
| DGCC13614 | Pediococcus pentosaceus | 918 | 1254 |
| 13391 | Lacticaseibacillus paracasei | 1146 | 1285 |
| 7391 | Lactiplantibacillus plantarum | 1039 | 1390 |
| TCF071MRS-C11 | Lactiplantibacillus pentosus | 1058 | 1612 |
| DGCC5799 | Pediococcus pentosaceus | 1144 | 1670 |
| TCF059MRS-A1 | Lactiplantibacillus pentosus | 1234 | 2674 |
| 12684 | Lacticaseibacillus paracasei | 1374 | 2814 |
| 12700 | Lacticaseibacillus paracasei | 1440 | 2880 |
| DGCC1246 | Lactiplantibacillus plantarum | 1440 | 2880 |
| 6518 | Levilactobacillus brevis | 1440 | 2880 |
| 6519 | Levilactobacillus brevis | 1440 | 2880 |
| 6520 | Levilactobacillus brevis | 1440 | 2880 |
| 6522 | Levilactobacillus brevis | 1440 | 2880 |
| DGCC13624 | Levilactobacillus brevis | 1440 | 2880 |
| 6506 | Levilactobacillus brevis | 1440 | 2880 |
| 6507 | Levilactobacillus brevis | 1440 | 2880 |
| 6508 | Levilactobacillus brevis | 1440 | 2880 |
| 7395 | Levilactobacillus brevis | 1440 | 2880 |
| 6509 | Levilactobacillus brevis | 1440 | 2880 |
| 6513 | Levilactobacillus brevis | 1440 | 2880 |
| 6514 | Levilactobacillus brevis | 1440 | 2880 |
| 6516 | Levilactobacillus brevis | 1440 | 2880 |
| 7638 | Pediococcus pentosaceus | 1440 | 2880 |
| TCF003-A1 | Lactiplantibacillus pentosus | 1440 | 2880 |
| TCF054MRS-A2 | Lactiplantibacillus pentosus | 1440 | 2880 |
| TCF048MRS-G6 | Limosilactobacillus fermentum | 1440 | 2880 |
| TCF079MRS-A2 | Limosilactobacillus fermentum | 1440 | 2880 |
| DGCC12587 | Limosilactobacillus fermentum | 1440 | 2880 |
| TCF083MRS-A9 | Limosilactobacillus fermentum | 1440 | 2880 |
| TCF152MRS-A12 | Limosilactobacillus fermentum | 1440 | 2880 |
| TCF152MRS-C8 | Limosilactobacillus fermentum | 1440 | 2880 |
| DGCC12605 | Limosilactobacillus fermentum | 1440 | 2880 |
| GL-14-F5 | Limosilactobacillus fermentum | 1440 | 2880 |
| TCF313MRS_A1?? | Limosilactobacillus fermentum | 1440 | 2880 |
| TCF082-A4 | Lactiplantibacillus pentosus | 1701 | 3141 |

The average TpH 6.00 was 434 minutes with a standard deviation of 32 minutes for Lactococcus lactis species and the average TpH 6.00 was 1040 minutes with a standard deviation of 345 minutes for the other lactic acid bacteria species tested. The average TpH5.50 was 543 minutes with a standard deviation of 46 minutes for Lactococcus lactis species and the average TpH 5.50 was 1693 minutes with a standard deviation of 639 minutes for the other lactic acid bacteria species tested. On average, Lactococcus lactis species acidified pea protein preparations approximately 2-3 times faster than the other lactic acid bacteria tested.

These results demonstrate that Lactococcus lactis species are useful for pea preparation acidification.

### Example 2: Acidification Test of Lactococcus lactis Strains in Preparation based on Soy Milk

The acidification kinetics of soy milk fermented with different lactic acid bacteria were assessed.

A commercial soy milk (Bjorg brand) was used as raw material for fermentation test. It is a UHT drink, containing 3.9% of protein. 2% of glucose is added during the preparation.

Before inoculation, the soy milk was warmed to 37° C. The inoculation rate was 1E6 cfu/ml for each bacterial stain tested. The pH of the soy milk was monitored during fermentation at 37° C. using an online pH measurement device (Abscia from Absciss Instrumentation Scientifique) and the time to reach a pH of 6.00 (TpH 6.00 in minutes) and 5.5 (TpH 5.50 in minutes) was determined.

The lactic acid bacteria tested included biodiverse Lactococcus lactis (28 strains) Streptococcus thermophilus (6 strains), Lacticaseibacillus paracaseï (23 strains), Lactiplantbacillus plantarum (7 strains), Lactiplantbacillus pentosus (15 strains), Levilactobacillus brevis (14 strains), Limosilactobacillus fermentum (9 strains), and Pediococus pentosaceus (3 strains). Table 2 shows the time to reach pH 6.00 (T PH 6.00 in minutes) and pH 5.5 (TpH 5.50 in minutes) for each of the 105 strains tested.

TABLE 2
TpH 6.00 an T PH 5.5 for 105 different Lactic acid
bacteria strain measured during soy milk fermentation,
ranked from the smallest to the largest TpH 5.50

| | | tpH | TpH |
|---|---|---|---|
| | | 6.00 | 5.5 |
| ID number | species | (Min) | (Min) |
| DGCC8006 | Streptococcus thermophilus | 182 | 211 |
| DGCC7710 | Streptococcus thermophilus | 212 | 253 |
| DGCC7919 | Streptococcus thermophilus | 246 | 281 |
| DGCC6297 | Streptococcus thermophilus | 246 | 291 |
| DGCC6297 | Streptococcus thermophilus | 252 | 299 |
| DGCC7666 | Streptococcus thermophilus | 262 | 307 |
| DGCC7693 | Streptococcus thermophilus | 266 | 315 |
| DSM33928 | Lactococcus lactis | 309 | 360 |
| DGCC12657 | Lactococcus lactis | 329 | 385 |
| DGCC12667 | Lactococcus lactis | 337 | 386 |
| DGCC2631 | Lactococcus lactis | 341 | 389 |
| DGCC12663 | Lactococcus lactis | 328 | 392 |
| DSM33925 | Lactococcus lactis | 338 | 392 |
| DSM33924 | Lactococcus lactis | 322 | 394 |
| DGCC12658 | Lactococcus lactis | 332 | 396 |
| DGCC12665 | Lactococcus lactis | 334 | 397 |
| DGCC12689 | Lactococcus lactis | 343 | 406 |
| DSM33926 | Lactococcus lactis | 344 | 414 |
| DGCC12673 | Lactococcus lactis | 357 | 415 |
| DGCC12678 | Lactococcus lactis | 357 | 421 |
| DGCC12654 | Lactococcus lactis | 350 | 425 |
| DGCC12671 | Lactococcus lactis | 376 | 437 |
| DGCC12686 | Lactococcus lactis | 370 | 437 |
| DGCC12685 | Lactococcus lactis | 384 | 438 |
| DGCC7283 | Lactococcus lactis | 391 | 443 |
| DGCC12662 | Lactococcus lactis | 382 | 444 |
| DGCC12691 | Lactococcus lactis | 384 | 447 |
| DGCC12668 | Lactococcus lactis | 391 | 459 |
| DSM33927 | Lactococcus lactis | 387 | 462 |
| DGCC 13614 | Pediococcus pentosaceus | 364 | 464 |
| DGCC12676 | Lactococcus lactis | 391 | 469 |
| DGCC12660 | Lactococcus lactis | 395 | 472 |
| DGCC12675 | Lactococcus lactis | 414 | 477 |
| DGCC12682 | Lactococcus lactis | 430 | 483 |
| TCF151MRS-A11 | Lactiplantibacillus pentosus | 429 | 500 |
| DGCC12651 | Lactococcus lactis | 417 | 503 |
| DGCC12688 | Lactococcus lactis | 444 | 510 |
| 14655 | Lacticaseibacillus paracasei | 445 | 521 |
| DGCC13613 | Lactiplantibacillus plantarum | 447 | 542 |
| DGCC5799 | Pediococcus pentosaceus | 445 | 557 |
| DGCC 13615 | Lacticaseibacillus paracasei | 494 | 560 |
| 14694 | Lacticaseibacillus paracasei | 500 | 569 |
| 13394 | Lacticaseibacillus paracasei | 512 | 583 |
| DGCC13620 | Lacticaseibacillus paracasei | 524 | 589 |
| 12726 | Lactiplantibacillus plantarum | 491 | 591 |
| SC-20-H1 | Lactiplantibacillus pentosus | 517 | 593 |
| TCF077MRS-A2 | Lactiplantibacillus pentosus | 524 | 596 |
| 7681 | Lacticaseibacillus paracasei | 541 | 606 |
| DGCC4715 | Lactiplantibacillus plantarum | 484 | 607 |
| 7387 | Lactiplantibacillus plantarum | 494 | 615 |
| 14693 | Lacticaseibacillus paracasei | 531 | 615 |
| TCF156MRS-A1 | Lactiplantibacillus pentosus | 567 | 617 |
| TCF059MRS-A1 | Lactiplantibacillus pentosus | 512 | 618 |
| DGCC12587 | Limosilactobacillus fermentum | 413 | 619 |
| 15338 | Lactiplantibacillus plantarum | 504 | 619 |
| DGCC11936 | Lactiplantibacillus pentosus | 519 | 620 |
| 13889 | Lacticaseibacillus paracasei | 528 | 624 |
| 7682 | Lacticaseibacillus paracasei | 553 | 626 |
| TCF149MRS-A1 | Lactiplantibacillus pentosus | 546 | 644 |
| 12689 | Lacticaseibacillus paracasei | 572 | 647 |
| SC-26-H3 | Lactiplantibacillus pentosus | 598 | 651 |
| SC-19-D3 | Lactiplantibacillus pentosus | 573 | 652 |
| 15321 | Lacticaseibacillus paracasei | 585 | 653 |
| DGCC1246 | Lactiplantibacillus plantarum | 552 | 654 |
| 7391 | Lactiplantibacillus plantarum | 539 | 657 |
| 12700 | Lacticaseibacillus paracasei | 561 | 659 |
| 14687 | Lacticaseibacillus paracasei | 594 | 668 |
| 7638 | Pediococcus pentosaceus | 579 | 670 |
| 14705 | Lacticaseibacillus paracasei | 597 | 675 |
| 7393 | Lacticaseibacillus paracasei | 590 | 682 |
| 7669 | Lacticaseibacillus paracasei | 600 | 684 |
| TCF071MRS-C11 | Lactiplantibacillus pentosus | 606 | 684 |
| TCF022LA-A12 | Lactiplantibacillus pentosus | 603 | 690 |
| 14634 | Lacticaseibacillus paracasei | 611 | 692 |
| 6516 | Levilactobacillus brevis | 601 | 698 |
| TCF082-A4 | Lactiplantibacillus pentosus | 627 | 719 |
| TCF049MRS-E9 | Lactiplantibacillus pentosus | 603 | 729 |
| TCF054MRS-A2 | Lactiplantibacillus pentosus | 625 | 737 |
| 7694 | Lacticaseibacillus paracasei | 666 | 758 |
| 13391 | Lacticaseibacillus paracasei | 683 | 758 |
| 11890 | Lacticaseibacillus paracasei | 680 | 770 |
| 6509 | Levilactobacillus brevis | 694 | 801 |
| TCF152MRS-A12 | Limosilactobacillus fermentum | 624 | 813 |
| 12684 | Lacticaseibacillus paracasei | 820 | 908 |
| 14679 | Lacticaseibacillus paracasei | 865 | 968 |
| TCF083MRS-A9 | Limosilactobacillus fermentum | 748 | 1095 |
| 6519 | Levilactobacillus brevis | 966 | 1153 |
| 6512 | Levilactobacillus brevis | 1170 | 1317 |
| 7395 | Levilactobacillus brevis | 1223 | 1349 |
| 6522 | Levilactobacillus brevis | 1210 | 1353 |
| TCF313MRS_A1?? | Limosilactobacillus fermentum | 1089 | 1461 |
| TCF152MRS-C8 | Limosilactobacillus fermentum | 1036 | 1481 |
| TCF079MRS-A2 | Limosilactobacillus fermentum | 743 | 2434 |
| DGCC12605 | Limosilactobacillus fermentum | 998 | 2438 |
| 6506 | Levilactobacillus brevis | 1440 | 2880 |
| 6507 | Levilactobacillus brevis | 1440 | 2880 |
| 6508 | Levilactobacillus brevis | 1440 | 2880 |
| 6513 | Levilactobacillus brevis | 1440 | 2880 |
| 6514 | Levilactobacillus brevis | 1440 | 2880 |
| 6518 | Levilactobacillus brevis | 1440 | 2880 |
| 6520 | Levilactobacillus brevis | 1440 | 2880 |
| TCF003-A1 | Lactiplantibacillus pentosus | 1440 | 2880 |
| GL-14-F5 | Limosilactobacillus fermentum | 1440 | 2880 |
| TCF048MRS-G6 | Limosilactobacillus fermentum | 1440 | 2880 |
| DGCC13624 | Levilactobacillus brevis | 1595 | 3035 |

The 28 strains belonging to Lactococcus lactis species were all in the top of the table 2, illustrating the short time needed for this species to achieve pH 5.5 in soy milk. Only Streptococcus thermophilus strains, species well known for soy milk adaptation, were faster in the pH range from initial soy milk pH to pH 5.5. All the strains form the other species tested exhibited higher TpH 5.50 than Lactococcus lactis strains.

These results demonstrate that Lactococcus lactis species are useful for soy milk acidification.

### Example 3: Growth Test of Lactococcus lactis Strains in Preparation based on Pea Proteins Preparation, Soy Milk and Oat Milk

A pea-based preparation was made using pea protein isolates TRUPRO 2000 (IFF). TRUPRO 2000powder was reconstituted at 4.5% (W/W) in water to reach e a final concentration of 3.7% protein. Glucose was added to the preparation to achieve a concentration of 2% glucose. The preparation was thoroughly mixed for 10 minutes with a Thermomix at a temperature of 80° C. prior to pasteurization at 90° C. for 90 minutes. The preparation was stored overnight at refrigerated temperature (ie. 4° C.).

A commercial soy milk (Bjorg brand) was used as raw material for fermentation test. It is a UHT drink, containing 3.9% of protein. 2% of glucose is added during the preparation.

Oat milk was made with oat flour, containing about 1.35% of protein and treated enzymatically (Foodpro Alt & Foodpro CGL). This preparation had been sterilized by using Ultra high temperature sterilization mode.

Before inoculation, the 3 preparations were warmed to 37° C. The inoculation rate was 1E6 5 cfu/ml for each bacterial stain tested belonging to Lactococcus lactis species. After 6 hours of incubation the concentration in CFU/g of plant-based preparation had been measured by using standard plate count technique for Lactococcus lactis, M 17—Lactose agar incubated 48h at 37° C. in anaerobiosis. Table 3 reports the concentration in CFU/g for each strain in each plant-based preparation

TABLE 3
CFU concentration of 28 Lactococcus lactis strains
after 6 h of fermentation at 37° C. in Oat
milk, pea protein preparation and soy milk.

| Strain | Oat - Cfu/ml | Pea - Cfu/ml | Soy - Cfu/ml |
|---|---|---|---|
| DSM33925 | 3.15E+08 | 2.11E+08 | 3.60E+08 |
| DGCC12651 | 6.80E+08 | 2.52E+08 | 5.90E+08 |
| DSM33926 | 6.45E+08 | 2.07E+08 | 3.25E+08 |
| DGCC12654 | 9.40E+08 | 3.05E+08 | 9.70E+08 |
| DGCC12657 | 7.95E+08 | 2.11E+08 | 6.95E+08 |
| DGCC12658 | 4.15E+08 | 2.80E+08 | 6.45E+08 |
| DGCC12660 | 2.35E+08 | 3.70E+08 | 3.70E+08 |
| DGCC12662 | 8.90E+08 | 2.80E+08 | 5.45E+08 |
| DGCC12663 | 7.95E+08 | 3.90E+08 | 6.30E+08 |
| DGCC12665 | 6.80E+08 | 2.52E+08 | 5.90E+08 |
| DGCC12667 | 7.75E+08 | 3.05E+08 | 9.70E+08 |
| DGCC12668 | 5.40E+08 | 2.60E+08 | 5.70E+08 |
| DSM33927 | 7.75E+08 | 2.40E+08 | 4.45E+08 |
| DGCC12671 | 7.45E+08 | 2.50E+08 | 5.25E+08 |
| DGCC12673 | 7.80E+08 | 2.60E+08 | 9.90E+08 |
| DGCC12675 | 6.80E+08 | 1.54E+08 | 6.85E+08 |
| DGCC12676 | 9.40E+08 | 2.90E+08 | 7.62E+08 |
| DGCC12678 | 3.15E+08 | 2.11E+08 | 3.60E+08 |
| DSM33928 | 7.95E+08 | 2.40E+08 | 6.95E+08 |
| DGCC12682 | 7.05E+08 | 2.30E+08 | 4.50E+08 |
| DGCC12685 | 9.15E+08 | 3.70E+08 | 7.15E+08 |
| DGCC12686 | 5.60E+08 | 2.20E+08 | 6.00E+08 |
| DGCC12688 | 5.70E+08 | 2.07E+08 | 3.25E+08 |
| DGCC12689 | 7.95E+08 | 2.40E+08 | 6.95E+08 |
| DGCC12691 | 3.15E+08 | 2.11E+08 | 3.60E+08 |
| DSM33924 | 5.08E+08 | 1.90E+08 | 6.00E+08 |
| DGCC7283 | 2.30E+08 | 1.73E+08 | 2.60E+08 |
| DSM33929 | 5.80E+08 | 1.00E+08 | 7.00E+08 |

After 6 hours of fermentation, for all the strains, the concentrations were at a minimum of 1.5 E8 cfu/ml and a maximum of 9.9 E8 cfu/ml in the three different plant-based preparations.

Results demonstrated the outstanding ability of Lactococcus lactis species to grow in plant-based preparations.

### Example 4: Combination of Lactococcus lactis Strains with Danisco Vege Starter Cultures for Soy Milk Fermentation and for Oat Milk Fermentation

A soy milk has been prepared by adding to a commercial soy milk, 1% Dextrose, 0.1% Pectin SY715 (Danisco) and 0.05% of salt. This is a classic recipe for manufacturing dairy alternative yoghurt based on soy.

An oat milk has been prepared by using an Oat syrup (Natu oat 62, Meurens) diluted 10 times as a basis and adding 4% VEGEDAN YO (Danisco) and 0.05% salt. This is a classic recipe for manufacturing dairy alternative yoghurt based on oat.

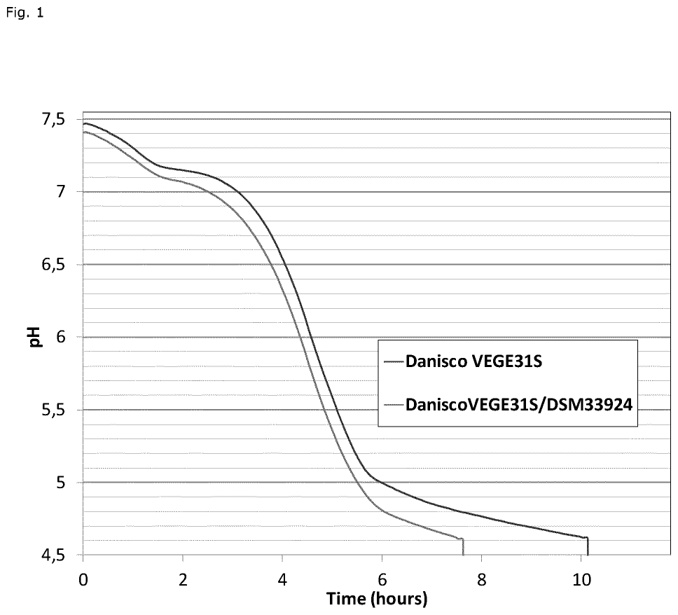

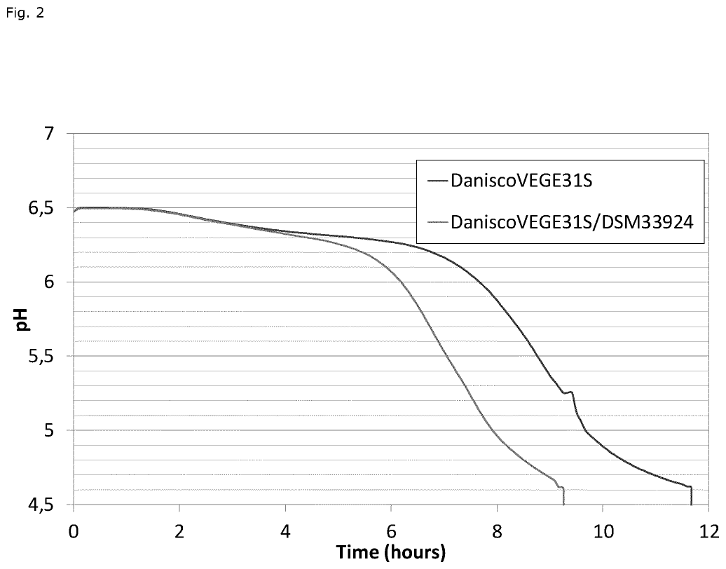

The inoculation rate of commercial starter cultures Danisco®VEGE 31S, composed of a mix of Streptococcus thermophilus and Lactobacillus delbrueckii bulgaricus strains, was 20 DCU/100L for soy and oat milk. In combination with this commercial starter culture, a Lactococcus lactis strain (DSM33924) was used at an inoculation level of 1.10E12 cfu/100L. The evolution of pH during fermentation is reported on FIG. 1 and FIG. 2.

The addition of Lactococcus lactis DSM33924 to the commercial starter culture Danisco®Vege was leading to a clear acceleration of acidification kinetics for soy and oat milk. It was observed that to achieve the pH 4.60, which represented the pH to start the cooling of the yoghurt from 40° C. to 6° C., the addition of Lactococcus lactis DSM33924 reduced the time of 2.5 h for soy yoghurt and 2.4 h for oat yoghurt compared to the yoghurt produced with Danisco®VEGE31S alone. This result demonstrated a clear advantage to use Lactococcus lactis species during yoghurt dairy alternative production to increase industrial yoghurt plant productivity.

### Example 5: Combination of Lactococcus lactis Strains with Danisco Vege Starter Cultures for Soy Milk Fermentation

A soy milk has been prepared by adding to a commercial soy milk, 2% Dextrose. The soy milk preparation is then heat treated at 95° C. for 6 minutes.

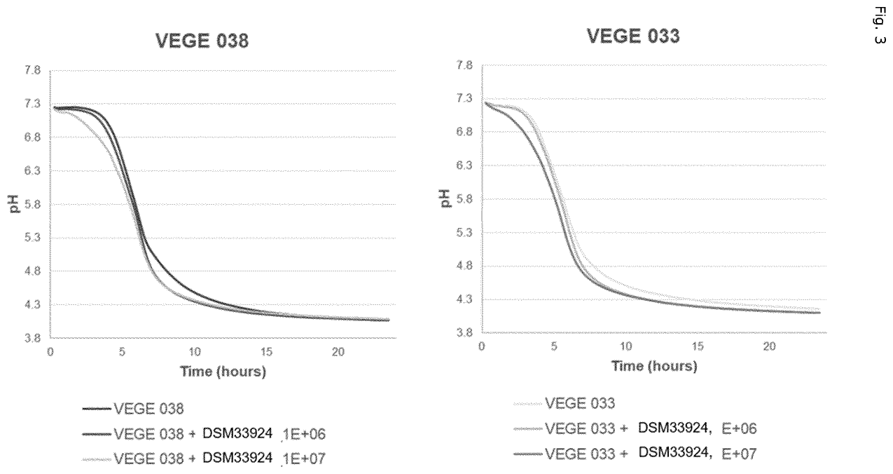

The inoculation rate of commercial starter cultures Danisco®VEGE 038, composed of a mix of Streptococcus thermophilus and Lactobacillus delbrueckii bulgaricus strains, was 20 DCU/100L. The inoculation rate of commercial starter cultures Danisco®VEGE 033, composed of a mix of Streptococcus thermophilus and Lactobacillus delbrueckii bulgaricus strains, was 20 DCU/100L. In combination with this commercial starter cultures, a Lactococcus lactis subsp. lactis strain (DSM33924) was used at an inoculation level of 1.10E11 cfu/100L or 1.10E12 cfu/100L. The inoculated milks were incubated at 37° C. The evolution of pH during fermentation is reported on FIG. 3. The addition of Lactococcus lactis DSM33924 to the commercial starter culture Danisco®Vege was leading to a clear acceleration of acidification kinetics. The lag phase of acidification and the time to achieve pH 4.60, which represented the pH to start the cooling of the yoghurt from 37° C. to 6° C., are markedly decreased (FIG. 3, and table 4). The table 4 reports the

TABLE 4
Time to achieve pH 4.60 in soy milk inoculated
with Danisco starter culture or Danisco starter
culture and DSM33924 strain, temperature 37° C.

| | TpH | Difference with |
|---|---|---|
| | 4.6 | and without addition |
| | (min) | of DSM33924 (min) |
| Dansico ®VEGE 038 20 DCU | 548 | |
| Dansico ®VEGE 038 20 DCU + 1E11 | 469 | −79 |
| cfu/100 L DSM33924 | | |
| Dansico ®VEGE 038 20 DCU + 1E12 | 469 | −79 |
| cfu/100 L DSM33924 | | |
| Dansico ®VEGE 033 20 DCU | 544 | |
| Dansico ®VEGE 033 20 DCU + 1E11 | 470 | −74 |
| cfu/100 L DSM33924 | | |
| Dansico ®VEGE 033 20 DCU+ 1E12 | 451 | −93 |
| cfu/100L DSM33924 | | |

The addition of DSM33924 was reducing the technological time of at least 1 hour and in one case 1.5 hour. This result demonstrated a clear advantage to use Lactococcus lactis species during yoghurt dairy alternative production to increase industrial yoghurt plant productivity.

1. A bacterial strain selected from the species of Lactococcus lactis, wherein the acidification kinetics of said bacterial strain in an aqueous preparation derived from a first plant containing 1-4% of protein from said plant and 2% glucose is characterized by:
an average time for reaching a pH of 6.00 less than about 510 min, measured as described in Assay I; and
an average time for reaching a pH of 5.5 less than about 660 min, measured as described in Assay I.

2. The bacterial strain according to claim 1, wherein the growth of said bacterial strain in an aqueous preparation derived from a second plant containing 1-4% of protein from said plant and 2% glucose measured as described in Assay II is characterized by:
an average of at least about 1.5×10⁸ cfu/ml after 6 hours of incubation in the aqueous preparation derived from a plant from an inoculation with 1×10⁶ cfu/ml.

3. The bacterial strain according to claim 1, wherein said bacterial strain originates from fermentation in an aqueous preparation derived from a third plant.

4. The bacterial strain according to claim 3, wherein two or three of said first, second and third plant are the same plant.

5. The bacterial strain according to claim 3, wherein two or three of said first, second and third plant are different plants.

6. The bacterial strain according to claim 3, wherein said first, second and/or third plant is independently selected from legumes, nuts, cereals and pseudo cereals, and oleaginous plants.

7. The bacterial strain according to claim 3, wherein said first, second and/or third plant is independently selected from pea, soy, and oat.

8. The bacterial strain according to claim 1, which is a protease-negative bacterial strain.

9. The bacterial strain according to claim 1, which is a strain of subspecies Lactococcus lactis subsp. lactis.

10. The bacterial strain according to claim 1, which is selected from the group consisting of:
(1) the DSM 33924 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(2) the DSM 33925 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(3) the DSM 33926 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(4) the DSM 33927 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(5) the DSM 33928 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH;
(6) the DSM 33929 bacterial strain deposited under the Budapest Treaty on 30 Jun. 2021 in the name of DuPont Nutrition Biosciences ApS, Denmark at the Leibniz-Institut DSMZ-Deutsche Sammlung von Mikroorganismen und Zellkulturen, GmbH; and
(7) a mutant bacterial strain of the DSM 33924 strain, the DSM 33925 strain, the DSM 33926 strain, the DSM 33927 strain, the DSM 33928 strain, or the DSM 33929 strain.

11. The bacterial strain according to claim 1, wherein the acidification kinetics is measured at a temperature below 39° C.

12. A composition, comprising or consisting of one or more cultures of a bacterial strain of claim 1.

13. The composition according to claim 12, wherein said composition further comprising at least one other lactic acid bacterium, and wherein said at least one other lactic acid bacterium is a different bacterial strain of the species Lactoccocus lactis, a different bacterial strain of the species Lactococcus cremoris, a different bacterial strain of the species Lactococcus hordinae, a bacterial strain of the species Streptococcus thermophilus, and/or a bacterial strain of the subspecies Lactobacillus delbrueckii subsp. bulgaricus, and/or a bacterial strain of the genus Bifidobacterium, and/or any combination thereof.

14. The composition according to claim 12, comprising a bacterial strain of the species Streptococcus thermophilus, and a bacterial strain of Lactobacillus delbrueckii subsp. bulgaricus.

15. (canceled)

16. The composition according to claim 12, which is in a liquid, a frozen or a dried-powder form.

17. The composition according to claim 12, further comprising at least one propionic bacterium.

18. The composition according to claim 12, comprising no other bacterial strain.

19.-22. (canceled)

23. A method for preparing a food or a feed product, wherein said method comprises putting into contact a plant-milk substrate, with or in the presence of a culture of a bacterial strain according to claim 1, fermenting said substrate, and obtaining said product.

24. (canceled)

25. A method for acidifying an aqueous preparation derived from a plant, which aqueous preparation contains 0-10%, protein from said plant and a sugar in an amount of 1-10%, the method comprising putting into contact the aqueous preparation, with or in the presence of a culture of a bacterial strain according to claim 1.

26.-27. (canceled)

28. A fermented food or feed product based on a plant, comprising a culture of a bacterial strain according to claim 1.

29.-31. (canceled)
